(19) United States
(12) Patent Application Publication
(10) Pub. No.: US 2011/0015227 A1
(43) Pub. Date: Jan. 20, 2011

(54) WARMING SENSATE COMPOSITION

(75) Inventors: Bernadita Desierto, Belle Mead, NJ (US); Anh Le, Princeton, NJ (US); Nicole Staniec, Ewing, NJ (US); John Zanone, Towaco, NJ (US)

Correspondence Address: WINSTON & STRAWN LLP;PATENT DEPARTMENT, 1700 K STREET, N.W., WASHINGTON, DC, 20006, US

(73) Assignee: FIRMENICH SA, Geneva (CH)

(21) Appl. No.: 12/933,314

(22) PCT Filed: Apr. 6, 2009
(86) PCT No.: PCT/IB2009/051435

§ 371 Date: Sep. 17, 2010

Related U.S. Application Data
(60) Provisional application No. 61/045,138, filed on Apr. 15, 2008.

(51) Int. Cl. A61K 31/4525; A61K 31/045; A61K 31/11; A61P 29/00; A23L 1/22

(52) U.S. Cl. 514/321; 514/729; 514/699; 426/536; 426/537; 426/534; 426/2

(57) ABSTRACT

The invention relates to a warming sensate composition of (i) a hot component, (ii) a cooling component, and (iii) optionally a bitter component. The weight ratio of (i):(ii) is from 1:3 to 1:100. If a solvent is provided in the composition, the solvent is present in an amount of less than 10 wt % ethanol, based on the total weight of the composition. Also, the bitter component, when used, is present in an amount of less than 0.15% by weight based on the total weight of the composition.

### TECHNICAL FIELD

The present invention relates to the field of flavor and taste. In particular, the invention relates to compositions and to edible products comprising the compositions which impart a warming sensation when consumed.

### BACKGROUND OF THE INVENTION

The particular sensations mediated by the trigeminal nerve, such as cooling, warming, pungent and salivating are becoming more appreciated by consumers in food, body care and perfumed products. These trigeminal compounds are capable of providing unique characteristics to a large variety of foods and they are even perceived on the skin of the body. Accordingly, U.S. Pat. No. 6,780,443, U.S. Pat. No. 6,890,567 and U.S. Pat. No. 6,899,901 (all to Takasago International Corporation) disclose compositions comprising a cooling sensate, a warming sensate and a tingling sensate, the purpose of which is to provide an initial strong sensation in the oral cavity, such as tingling or stinging.

It is also known to combine compounds known to possess flavor and/or sensate compounds to produce new active ingredients having altered properties. For example, WO 98/47482 discloses formulations for cough drops which include a physiological cooling agent.

None of these documents addresses the issue of providing a warming sensation in the mouth, throat and chest.

In the pharmaceutical industry, over the counter (“OTC”) type medicines, particularly analgesics such as cold relief products, and especially cough syrups, often contain ethanol, which not only acts as a solvent for the active ingredients but, more importantly for the consumer, delivers a mouth, throat and chest-warming sensation, which serves to reinforce the soothing effect delivered by the medicine. However, the use of ethanol in such products may not always be desirable. For instance, some consumers prefer to avoid its consumption for health, religious or other reasons and some consumers are known to suffer an allergic reaction to it and are consequently obliged to avoid its consumption. Therefore, it would be a clearly desirable consumer benefit to provide a product that achieves substantially the same warming sensation as provided by ethanol and so can replace, at least partly, the ethanol content therein.

U.S. Pat. No. 6,780,443, U.S. Pat. No. 6,890,567 and U.S. Pat. No. 6,899,901 do not address this issue and even disclose numerous compositions containing very high levels of ethanol.

In another aspect, the composition should preferably avoid delivering spicy- or off-notes since these will be undesirable in analgesic consumer products such as cough mixtures, where such spiciness may even serve to further irritate a sore throat.

Therefore, it is a preferred objective of the present invention to provide a warming effect devoid of strong spicy notes. In particular, it is an objective to provide a flavour sensation composition providing a clean, warming trigeminal effect capable of replacing substantially or entirely ethanol in a product, especially, an analgesic product.

In particular, it is an objective of the present invention to provide a composition in which the ingredients synergistically interact with each other to provide a warming sensation in a way that the sensation is perceived in not only the mouth but also the throat and the chest.

### SUMMARY OF THE INVENTION

Remarkably, the present inventors have found that a composition comprising a hot component and a cooling component in a specific ratio is capable of providing a clean warming sensation that is perceived not only in the mouth but also in the throat and chest, and so is effective as a replacement for ethanol where it is used to this effect.

Accordingly, the present invention provides, in a first aspect, a warming sensate composition comprising

(i) a hot component,

(ii) a cooling component,

(iii) optionally a bitter component, and

(iv) optionally a solvent for components (i) to (iii)

wherein the weight ratio of (i):(ii) is from 1:3 to 1:100 with the proviso that the bitter component (iii) is present at less than 0.15% by weight based on the total weight of the composition, and the solvent (iv) comprises less than 10 wt % ethanol, based on the total weight of the warming sensate composition.

In another aspect, the present invention provides the use of a composition as defined above to deliver a warming sensation to the mouth, throat and chest of a consumer.

In a further aspect, the invention provides a food product, an oral care product, a body care product or a pharmaceutical product comprising the warming sensate composition as defined above.

### DETAILED DESCRIPTION OF THE INVENTION

The composition of the present invention comprises a hot component and a cooling component wherein the weight ratio of hot component to cooling component is from 1:3 to 1:100.

The composition is preferably used in orally ingestible products. More preferably, it is used in pharmaceutical products, even more preferably analgesic preparations. The product may be provided in the form of a solid, liquid, gel, suspension or the like. Particularly preferred are syrups and liquid-centred throat lozenges, where ethanol is conventionally used to deliver a warming sensation to the mouth, throat and chest.

Nevertheless, it is envisaged that the composition of the invention may also be used in other consumer products where the warming sensation referred to above is desired. Therefore, the composition can also be used in beverages, which may be either alcoholic or non-alcoholic, chewing gums, confectionary, savoury foods and so on.

For instance, certain beverages are consumed warm or hot and so it may be desirable to achieve such a warming effect even in the absence of a means for heating the beverage. An example is mulled wine, an alcoholic beverage consumed warm to give a pleasant warming effect. By incorporating the composition of the invention, the level of alcohol can even be reduced whilst maintaining the desired warming effect.

### Hot Sensate Component

The composition according to the invention comprises a hot component consisting of one or more hot sensate compounds. By hot sensate compound, it is meant a compound which provides a hot, warming sensation when brought into contact with the oral cavity or the skin. In particular, the hot component provides a hot, warming sensation mediated by the trigeminal nerve. The hot compound is believed to stimulate primarily receptor TRPV1.

The hot compound is preferably a purified compound, but may also be provided in the form of an extract.

Examples of suitable hot sensate compounds include those having a structure according to the following formula:

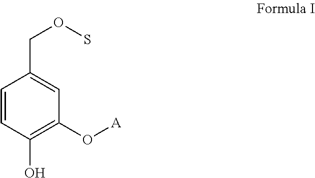

or an acceptable salt thereof wherein A is an unsubstituted, branched or straight-chained C₁-C₃ alkyl group and B is a hydrogen, or an unsubstituted, branched or straight-chained C₁-C₇ alkyl group. In one embodiment A represents a methyl group. In a preferred embodiment, A represents a methyl group and B represents a C₂-C₄ alkyl group. In a particularly preferred embodiment, the warming sensate is selected from vanillyl butyl ether and vanillyl ethyl ether. Vanillyl butyl ether is commercially available under the trade name Hotact® VBE (ex Takasago, Inc). Other hot sensate compounds suitable for use in the composition of the present invention include gingerol, vanillyl propyl ether, vanillyl pentyl ether, vanillyl hexyl ether, vanillyl butyl ether acetate, 4-(1-menthoxymethyl)-2-phenyl-1,3-dioxolan, 4-(1-menthoxymethyl)-2-(3′,4′-dihydroxyphenyl)-1,3-dioxolan, 4-(1-menthoxymethyl)-2-(2′-hydroxy-3′-methoxyphenyl)-1,3-dioxolan, 4-(1-menthoxymethyl)-2-(4′-methoxyphenyl)-1,3-dioxolan, 441-menthoxymethyl)-2-(3′,4′-methylenedioxyphenyl)-1,3-dioxolan, 4-(1-menthoxymethyl)-2-(3′-methoxy-4′-hydroxyphenyl)-1,3-dioxolan, red pepper oil, red pepper oleoresin, ginger oleoresin, nonylic acid vanillyl amide, jambu oleoresin, Zanthoxylum piperitum extract, sanshool I, sanshool II, sanshoamide, black pepper extract, chavicine, piperine and spilanthol. Other suitable hot sensate compounds are disclosed in, for example, U.S. Pat. No. 6,780,443, hereby incorporated by reference in its entirety. The hot component preferably excludes capsaicin since the spiciness perceived by the consumer of such an ingredient is not desirable in the present instance. If capsaicin is to be included, then it is preferably present at a level of less than 0.5% by weight based on the weight of the warming sensate composition.

### Cooling Sensate Component

The composition according to the invention further comprises a cooling component consisting of one or more cooling sensate compounds, with the proviso that, in the finished product, there is no noticeable mentholic taste. By cooling sensate compound, it is meant any compound which, when brought in contact with the skin or oral cavity, provides a cooling sensation, which is mediated by the trigeminal nerve. The cooling compound primarily activates the TRPM8 receptor.

The cooling component is preferably a purified compound, but may also be provided in the form of an extract.

Examples of suitable cooling sensate compounds include (3S,5R,6S,9R)-6-isopropyl-3,9-dimethyl-1,4-dioxaspiro[4.5]decan-2-one and (3S,5S,6S,9R)-6-isopropyl-3,9-dimethyl-1,4-dioxaspiro[4.5]decan-2-one (both marketed under the name cubebol, Firmenich SA), compounds having a structure according to the following formula:

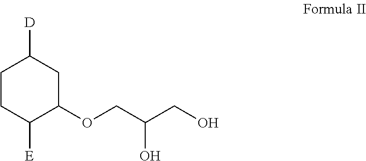

or salts thereof, wherein D is a straight chained or branched, unsubstituted C₁-C₄ alkyl or alkenyl group and E is a straight chained or branched, hydroxy-substituted or unsubstituted C₁-C₄ alkyl group, such as (3-1-menthoxypropane-1,2-diol), available commercially under the name Coolact® 10 (ex Takasago, Inc.), menthol, menthone, camphor, pulegol, isopulegol, cineol, mint oil, peppermint oil or fractions thereof, spearmint oil, eucalyptus oil, N-alkyl-p-menthane-3-carboxamide, 3-1-menthoxy-2-methylpropane-1,2-diol, p-menthane-3,8-diol, 2-1-menthoxyethane-1-ol, 3-1-menthoxypropane-1-ol, 4-1-menthoxybutane-1-ol, 1-(2-hydroxy-4-ethylcyclohexyl)-ethanone, menthyl 3-hydroxybutanoate, menthyl lactate, menthone glycerin ketal, 242-1-menthyloxyethyl)ethanol, menthyl glyoxylate, N-methyl-2,2-isopropylmethyl-3-methylbutanamide, menthyl 2-pyrrolidone-5-carboxylate, monomenthyl succinate, alkali metal salts of monomenthyl succinate, and alkali earth metal salts of monomenthyl succinate, monomenthyl glutarate, alkali metal salts of monomenthyl glutarate, alkali earth metal salts of monomenthyl glutarate, N-[[5-methyl-2-(1-methylethyl)cyclohexyl]carbonyl]glycine, p-menthane-3-carboxylic acid glycerol ester, Menthol propylene glycol carbonate; Menthol ethylene glycol carbonate, and 2-(1-methylpropyl)-1-cyclohexanone (Freskomenthe®, Givaudan). Other cooling sensates are disclosed in U.S. Pat. Nos. 7,030,273 and 6,780,443, which are hereby incorporated by reference in their entirety.

If it is desired to use, as the cooling sensate, a compound which delivers a mentholic flavor, it is essential that this is present in the finished product at below the threshold at which a consumer is capable of detecting the menthol flavor. For instance, it is preferred that menthol or mentholic flavor compound is present at a level of 2% by weight or less of the warming sensate composition. This is because the perception by a consumer of the throat and chest warming effect, which is achievable by the combination of cooling, heating and optionally tingling components, is significantly diminished when the consumer also perceives a menthol flavor. Thus, in one embodiment, the composition may be substantially, more preferably entirely free of menthol and menthol derivatives.

### Bitter Component

Whilst not essential, it has been found that the warming sensation in the throat and chest can be accentuated by the presence of low levels of a bitter component. Accordingly, it is preferred that the composition of the invention comprises a bitter component. The bitter component is a compound or, more generally, an extract having bitter organoleptic properties. Bitterness is one of five recognized basic tastes (besides sweet, salty, sour and umami) and is mediated by taste buds at the back of the tongue. It can readily be attributed to compounds, ingredients, for example. In a preferred embodiment of the invention, the bitter component is selected from bitter triterpenes, glucosides of monoterpenes, sesquiterpenic lactones, humulone, lupulone, flavonones, quinines, and mixtures of these. Examples for bitter plant extracts include Quassia extract (also called bitter ash extract, FEMA No. 2971), quinquina extract, chamomile oils (FEMA No. 2272 to 2274), gentian root extract (FEMA No. 2506), hops extracts and oils (FEMA No. 2578 to 2580, artichoke leaves oil and so on.

Surprisingly, the accentuating effect can be achieved with a very low level of the bitter component. For instance, it may be present at levels below 0.15% by weight of the warming sensate composition, even below 0.1%, or even below 0.08%. The use of very low levels is advantageous since the risk of a consumer perceiving bitterness is dramatically reduced.

### Solvent

The composition of the present invention preferably comprises at least one solvent, or a mixture of solvents, to dissolve the different components of the composition according to the invention with the proviso that the solvent comprises less than 10 wt % ethanol based on the total weight of the sensate composition. Preferably, the solvent comprises less than 5 wt %, more preferably less than 2 wt %, most preferably less than 1 wt % ethanol, or is even substantially free of ethanol. Solvents of current use for the preparation of the sensate composition include, for instance, benzyl alcohol, propylene glycol, triacetine, neobee, vegetable oils or limonene. Propylene glycol is most preferred. A mixture of solvents may be useful to dissolve hydrophobic and hydrophilic components in the same solution. In this case, ethanol may be present but at the very low levels stipulated above. For example, the components of the present invention may be in a solution comprising 20-99.9%, preferably 40-99.9%, more preferably 80-99.9%, even more preferably 90-99.9% of solvent. In the context of the present invention, percentages are percentages by weight, unless otherwise indicated. Similarly, if proportions are indicated as parts, parts by weight are meant.

The composition of the invention may further comprise other ingredients typically used in flavor compositions. For example, the composition may comprise further flavours. The composition may also comprise adjuvants that allow the composition to meet technical requirements, such as stability or tonality persistence. Today, the range of products types and product formulations that are flavoured has become so extensive and subjected to frequent changes that an approach made on a product-by-product basis and on the definition for each case of the adjuvants that can be used is impractical. This is why a list of adjuvants currently used in flavouring formulations is not given here. However, a skilled person in the art, for example a flavourist is capable of choosing these ingredients as a function of the product to be flavoured and of the nature of the flavouring ingredients contained in the formulation.

The sensate composition comprises a hot component comprising one or more hot ingredients and a cooling component comprising one or more cooling ingredients wherein the weight ratio of hot to cooling components is from 1:3 to 1:100, more preferably from 1:3 to 1:50, most preferably from 1:7 to 1:35.

Below 1:3, it has been found that the chest warming effect is not achieved whereas above 1:100, the amount of cooling component negates the warming effect and so it cannot be clearly perceived. Furthermore, above a ratio of 1:100, the presence of even small amounts of “mentholic” compounds may give rise to a noticeable mentholic flavor.

Furthermore, it is preferred that the total amount of cooling and hot components is at least 0.3 wt %, based on the total weight of the product in which it is present. Below, this level it is found that the chest warming effect is barely noticeable. By contrast, it is preferred that the total amount of cooling and hot components does not exceed 1 wt % based on the total weight of the product in which it is present since this is found to have an undesirable stinging sensation.

The composition of the invention can be made by simply mixing all components of the invention with a solvent at room temperature, at the weight-ratios mentioned above.

In one aspect, the present invention provides of a composition according to the invention to provide a warming flavor to a consumable product, more preferably to providing a throat and chest-warming flavor.

In another aspect, the present invention provides a method for imparting chest and throat warming sensation to a consumable product, the method comprising the steps of adding to the consumable product the composition of the present invention.

In the above uses and method, the composition of the invention may be used directly as it is or added to food products as described below.

The composition of the invention may directly be added to a consumable product at the stage where flavours are generally added. If convenient, a liquid composition may be encapsulated in a matrix, such as a carbohydrate, a phospholipid, or a protein matrix, such as a gelatine matrix, for example, or any bioactive matrix, in order to provide a shelf-stable, dry form of the flavor composition of the invention.

Therefore, in an embodiment, the present invention provides a delivery system comprising the composition of the present invention. Preferably, the delivery system is in the form of a particulate composition and/or a powder. For example, the composition may be encapsulated following standard procedures in spray-dried powders. For example, the composition may be encapsulated in a matrix, which is in a glassy state at room temperature. For example, the composition of the invention may be encapsulated in rod-like granules, the preparation of which is disclosed in U.S. Pat. No. 4,707,367.

The composition of the invention may be used in liquid products and/or in products that are solid. In a preferred embodiment, the product is a liquid since the chest and throat warming effect is found to be more readily achievable in such a product. The consumable product is preferably selected from the group consisting of a liquid pharmaceutical product, such as a liquid analgesic, a beverage, an alcoholic drink, a dairy product, a soy product, a soup, a dressing, a sauce, a dip, and the like.

In one aspect, the present invention provides an oral care product according to the invention. For example, the product may be a toothpaste, a tooth-gel, a mouthwash.

In another aspect, the present invention provides a pharmaceutical product comprising the flavor composition according to the invention. Examples of such products are syrups, lozenges, chewing gums, and drugs provided in the form of tablets or capsules.

The examples below further illustrate the invention without limiting its scope. Samples according to the invention are denoted by a number and comparative samples are denoted by a letter. All amounts are denoted in % by weight unless otherwise stated.

### Example 1

### Preparation of a Sensate Composition

In preparing the sensate compositions, the ingredients described in table 1 were used.

TABLE 1

| Ingredient | Characteristics | Origin |
|---|---|---|
| Anethol | Cooling | Polarome, Inc |
| Menthol Nat. | Cooling | Aldrich |
| Mint Piperita | Cooling | Idaho, USA |
| Cooling Agent 10 | Cooling | Takasago, Inc |
| Coolact P | Cooling | Takasago, Inc |
| Hotact VBE | Hot | Takasago, Inc |
| WS3 | Cooling | Lyondell Chemical Company |
| WS23 | Cooling | Lyondell Chemical Company |
| Piperine | Hot | Aldrich |

Sample A was prepared by mixing together 5 parts Cooling agent 10, 5 parts Coolact P and 25 parts Hotact VBE in 965 parts propylene glycol solvent.
Sample B was prepared by mixing together 100 parts menthol, 125 parts piperine, 125 parts WS3, 375 parts Hotact VBE in 9275 parts propylene glycol solvent.
Sample C was prepared by mixing together 55 parts anethol polarome, 167 parts menthol and 333 parts mint piperita in 445 parts triacetine.
Sample D was prepared by mixing 100 parts WS23 in 900 parts propylene glycol solvent. Compositions E, F and 1 to 4 were prepared by mixing various quantities of samples A, B, C and D together with a defined amount of an aqueous 5% sucrose solution. The amounts are given in the following table.

TABLE 2

| | Sucrose | | | | | | Wt Ratio |
|---|---|---|---|---|---|---|---|
| Composition | soln. | A | B | C | D | Other | Hot:Cool |
| E | 99.80 | 0.05 | 0.05 | — | — | 0.10* | 2.14:1 |
| F | 99.72 | 0.08 | 0.08 | — | 0.12 | — | 1:2.43 |
| 1 | 99.70 | 0.05 | 0.05 | 0.20 | — | — | 1:30 |
| 2 | 99.63 | 0.08 | 0.05 | 0.08 | — | 0.16* | 1:9.8 |
| 3 | 99.64 | 0.08 | 0.08 | — | 0.20 | — | 1:3.8 |
| 4 | 99.36 | 0.12 | 0.08 | 0.20 | 0.24 | — | 1:19.6 |

*Cherry Flavour (ex Firmenich, code 057679A)

The samples were then evaluated by a team of 6 trained panelists. Each panelist was asked to describe the effect on the tongue, in the mouth, in the throat and in the chest. The results are given in the following table:

TABLE 3

| Sample | Evaluation |
|---|---|
| E | Cherry, sweet |
| | Hot in the mouth and biting (tingling) on the tongue |
| | No chest warming |
| F | No chest warming |
| | Weak |
| 1 | Gradually perceived chest warming |
| 2 | Cherry, sweet and cooling |
| | Hot in the mouth and biting (tingling) on the tongue |
| | Chest warming |
| 3 | Good chest warming |
| 4 | Chest warming |

The results demonstrate that the desired warming effect in the chest is achieved when the weight ratio of hot to cool ingredients is 1:3 or greater. The results also demonstrate that the effect is not due to the presence of the propylene glycol solvent.

### Example 2

### Candy Comprising a Chest-Warming Sensate Composition

Various samples of syrups are prepared with different warming compositions according to the procedure below.

Isomalt (100 g) and water (30 g) are mixed and heated to 160-165° C. in a copper pan. At 165° C., the copper pan is removed from the gas frame and placed in a warm water bath (40° C.). After a few seconds, the copper pan is removed and the temperature checked. When the temperature reaches 135° C., the mixture comprising is further complemented with 1% of an aqueous solution containing Aspartame (10%) and Acesulfame-K (5%). Thereafter, sample 1 is added to provide a composition comprising a total amount of hot and cooling ingredients of 0.3 wt % based on the total weight of syrup. The cooked mass is then poured at ambient temperature (at less than 40% relative humidity) into appropriate Teflon® molds and allowed to cool to provide candies comprising the chest warming composition.

### Example 3

### Sugar-Free Chewing Gum Comprising the Warming Composition of the Invention

Sugar-free chewing gums are prepared by the following standard procedure: Crystalline sorbitol, Acesulfame-K and aspartame are blended in a Turbula blender. Half the blend is mixed with a pre-warmed Sierra gum base (Cafosa) in a Winkworth sigma-blade mixer at 50-55° C. for 2 minutes. The remaining powder blend is then added along with a humectant syrup (Lycasin® 80/55, Sorbit®, glycerin) and mixed for a further 7 minutes. Finally, the sample 2 is added and mixed for one minute at a dosage of 0.35% based on the total weight of chewing gum.

### Example 4

### Savory Applications Using an Invention's Composition

A dry blended mixture flavor was prepared according to the following composition:

### Component

| | |
|---|---|
| Salt | 20.00% |
| MSG | 5.00% |
| Ribonucleotide | 0.20% |
| Sugar | 10.00% |
| Yeast Extract Powder | 10.00% |
| Chili Powder | 3.00% |
| Onion Powder | 2.00% |
| Garlic Powder | 2.00% |
| Tomato Powder | 5.00% |
| Whey Powder | 42.30% |
| Paprika Oleoresin | 0.08% |
| Neobee | 0.22% |
| Sample 3 | 0.20% |
| | 100.00% |

1-10. (canceled)

11. A warming sensate composition comprising:
(i) a hot component,
(ii) a cooling component,
(iii) optionally, a bitter component, and
(iv) optionally, a solvent for components (i) to (iii);
wherein the weight ratio of (i):(ii) is from 1:3 to 1:100 with the provisos that the bitter component (iii) when included, is present in an amount of less than 0.15% by weight based on the total weight of the composition, and the solvent (iv), when included, comprises less than 10 wt % ethanol, based on the total weight of the warming sensate composition.

12. The composition according to claim 11, wherein the weight ratio of (i) to (ii) is from 1:7 to 1:35.

13. The composition according to claim 11, wherein the total amount of (i) and (ii) is at least 0.3 wt %, based on the total weight of any consumable product in which it is present.

14. The composition according to claim 11, wherein the total amount of (i) and (ii) is no greater than 1 wt %, based on the total weight of any consumable product in which it is present.

15. The composition according to claim 11, in which the hot component agent is selected from the group consisting of piperine, pelargonyl vanillyl amide, vanillyl butyl amide, vanillin butyl ether, eugenol, gingerol, polygodial, shogoal, galangal acetate, capsaicin (N-(4-hydroxy-3-methoxybenzyl)-8-methylnonanamide and/or (6E)-N-(4-hydroxy-3-methoxybenzyl)-8-methyl-6-nonenamide), capsaicin analogs and mixtures comprising two or more of these.

16. The composition according to claim 11, in which the cooling component is selected from the group consisting of menthol, menthyl succinate, menthyl lactate, p-menthane-3,8-diol, 8-p-menthen-3-ol, 3-(3′-P-menthanyloxy)-1,2-propanediol, menthone glycerol ketal, 2-(1-methylpropyl)-1-cyclohexanone, N-ethyl-3-P-menthanecarboxamide, aspartic acid, N-(4-hydroxy-3-methoxybenzyl)nonanamide, 545-(1,3-benzodioxol-5-yl)-1-(1-piperidinyl)-2,4-pentadien-1-one, 6-isopropyl-3,9-dimethyl-1,4-dioxaspiro[4.5]decan-2-one, 2-isopropyl-N,2,3-trimethylbutanamide, and, 7-isopropyl-4,10-dimethyl-tricyclo[4.4.0.0(1,5)]decan-4-ol, 3-menthyl-3,6-dioxaheptanoate, 3-menthyl methoxyacetate, 3-menthyl-3,6,9-trioxadecanoate, 3-menthyl-(2-hydroxyethoxy)acetate, menthyl-11-hydroxy-3,6,9-trioxadecanoate, 3S,5R,6S,9R)-6-isopropyl-3,9-dimethyl-1,4-dioxaspiro[4.5]decan-2-one, (3S,5S,6S,9R)-6-isopropyl-3,9-dimethyl-1,4-dioxaspiro[4.5]decan-2-one, 2,3-dihydroxypropyl (1R,2S,5R)-2-isopropyl-5-methylcyclohexane carboxylate, (3S,5R,6S,9R)-6-isopropyl-3,9-dimethyl-1,4-dioxaspiro[4.5]decan-2-one, (3S,5S,6S,9R)-6-isopropyl-3,9-dimethyl-1,4-dioxaspiro[4.5]decan-2-one, (1R,2S,5R)—N-(tert-butyl)-2-isopropyl-5-methyl-cyclohexanecarboxamide and mixtures comprising two or more of these.

17. A method of delivering a warming sensation to the mouth, throat or chest of a consumer which comprises administering to the consumer one or more of a warming sensate composition as defined in claim 11 or a food product, an oral care product, a body care product or a pharmaceutical product containing the warming sensate composition.

18. The method according to claim 17, wherein the warming sensate composition is orally administered to the consumer in the form of a liquid pharmaceutical product, a beverage, an alcoholic drink, a dairy product, a soy product, a soup, a dressing, a sauce or a dip.

19. A food product, an oral care product, a body care product or a pharmaceutical product containing the warming sensate composition according to claim 11.

20. The product according to claim 19, in the form of a liquid pharmaceutical product, a beverage, an alcoholic drink, a dairy product, a soy product, a soup, a dressing, a sauce or a dip.

21. The product according to claim 20 wherein the liquid pharmaceutical product is a liquid analgesic preparation.

22. The product according to claim 19, wherein the total amount of (i) and (ii) is at least 0.3 wt %, based on the total weight of the product.

23. The product according to claim 19, wherein the total amount of (i) and (ii) is no greater than 1 wt %, based on the total weight of the product.
